(19) United States
(12) Patent Application Publication
(10) Pub. No.: US 2006/0178591 A1
(43) Pub. Date: Aug. 10, 2006

(54) Methods and systems for real time breath rate determination with limited processor resources

(76) Inventor: Ralf Hans Hempfling, Ventura, CA (US)

Correspondence Address: WINSTON & STRAWN LLP, 1700 K STREET, N.W., WASHINGTON, DC, 20006, US

(21) Appl. No.: 11/285,609

(22) Filed: Nov. 21, 2005

Related U.S. Application Data
(60) Provisional application No. 60/629,464, filed on Nov. 19, 2004.

(51) Int. Cl. A61B 5/08; A61B 5/04

(52) U.S. Cl. 600/529; 600/509

(57) ABSTRACT

A method for recognizing occurrences of breaths in respiratory signals. The method includes receiving digitized respiratory signals that includes tidal volume signals, filtering the received respiratory signals to limit artifacts having a duration less than a selected duration, and recognizing breaths in the filtered respiratory signals. A breath is recognized when amplitude deviations in filtered tidal volume signals exceed a selected fraction of an average of previously determined breaths. This invention also include methods for recognizing breathes from electrocardiogram R-waves; computer methods having code for performing the methods of this invention; monitoring systems that monitor a subject and include local or remote computers or other devices that perform the methods of this invention.

### CROSS-REFERENCE TO RELATED APPLICATIONS

This application claims the benefit of U.S. Provisional Application No. 60/629,464, filed on Nov. 19, 2004, the entire content of which is expressly incorporated herein by reference thereto.

### FIELD OF THE INVENTION

The present invention relates to processing physiological data from monitored subjects, and in particular provides methods for extracting breath rate on handheld-type systems using available computer resources.

### BACKGROUND OF THE INVENTION

Real-time ambulatory monitoring of physiological signs, such as heart rate (“HR”) and breath rate (“BR”), is important in a variety of situations. Such ambulatory monitoring systems are available and often include a handheld-type computer local to a monitored subject for buffering and retransmitting monitored data for later analysis. See, e.g., the LifeShirt™ from VivoMetrics, Inc. (Ventura, Calif.). It is advantageous that such a handheld-type computer also extract real-time physiological signs from monitored data, in particular breath rate and heart rate.

The more limited processing capabilities of handheld-type systems make such extraction more difficult in comparison to extraction using more capable remote server systems. For example, extraction methods for server systems with large and easily expandable processing capabilities often involve extensive filtering and other signal analysis operations which cannot be easily performed by the processing capacity available in handheld-type computers. Furthermore, to be useful, handheld-type extraction methods must solve additional challenges that include the following: available power and speed; real time processing with minimal latency; adapting processing to a wide range of monitored subjects and monitoring environments; extracting parameters accurately; in particular the minimizing the number of missed events and/or falsely identified events, such as breaths; and effectively removing motion artifacts that are likely in data from active subjects. Such methods for addressing these challenges are not known in the prior art.

### SUMMARY OF THE INVENTION

A preferred embodiment of the present invention is directed to method for recognizing occurrences of breaths in respiratory signals and suitable for handheld-type computers and other electronic devices. The method includes a first method including receiving digitized respiratory signals that include tidal volume signals, filtering the received respiratory signals to limit artifacts having a duration less than a selected duration, and recognizing breaths in the filtered respiratory signals. A breath is recognized when amplitude deviations in filtered tidal volume signals exceed a selected fraction of an average of previously determined breaths. Preferably, the method further includes determining a breath rate from the occurrences of breaths.

The selected fraction preferably varies in dependence on a subject activity level. Filtering the respiratory signals preferably includes filtering one or more respiratory signal samples by taking a median value of respiratory signal samples occurring during a selected duration. Preferably, the median value includes the respiratory signal sample being filtered. More preferably, filtering the respiratory signals further includes applying a linear low-pass filter to the signals. The selected duration preferably varies in dependence on a subject activity level that is determined from one or more high-pass filtered accelerometer signals. Preferably, the respiratory signals are detected using inductive plethysmographic size sensors disposed about the rib cage and/or abdomen of a monitored subject.

In one embodiment, the method of recognizing occurrences of breaths further includes a second method that includes recognizing breaths from variations in heart rate that are reflective of respiratory sinus arrhythmia. Preferably, the variations in heart rate are determined from R-wave signals recognized in an electrocardiographic signal. The recognition of R-waves preferably includes determining a signal-to-noise ratio by comparing two differently scaled moving averages of the received electrocardiographic signal, selecting signal maxima when electrocardiographic signal deviations exceed a selected signal-to-noise threshold, and recognizing R-waves from the selected signal maxima occurring in a selected temporal relationship to adjacent recognized R-waves. Additionally, the method can further include comparing one or more breaths recognized by the first method and one or more breaths recognized by the second method, and selecting one or more recognized occurrences of breaths from and in dependence on the compared breaths.

The method also preferably includes concurrently performing additional instances of the steps of receiving, filtering, and recognizing, wherein the selected fraction and/or the selected duration of each separate instance are different. One or more breaths recognized by the additional instances of the steps of receiving, filtering, and recognizing are then preferably compared, and one or more recognized occurrences of breaths are selected from and in dependence on the compared breaths.

The present invention is also directed to a computer memory having instructions for executing a method of recognizing occurrences of breaths. Preferably, the computer memory is operatively linked to a computer system such as a handheld-type computer.

The present invention is also directed to a method for recognizing R-waves in electrocardiographic signals. The method includes receiving a digitized electrocardiographic signal, determining a signal-to-noise ratio by comparing two differently scaled moving averages of the received electrocardiographic signal, selecting signal maxima when electrocardiographic signal deviations exceed a selected signal-to-noise ratio threshold, and recognizing R-waves from the selected signal maxima occurring in a selected temporal relationship to adjacent recognized R-waves. Preferably, the heart rate signal is filtered to remove minima therein. The method can also includes recognizing occurrence breaths in dependence on minima and/or maxima of the heart rate signal.

The present invention is also directed to a method for determining occurrences of breaths in physiological signals gathered from a monitored subject. The method includes performing at least one breath rate detection method, wherein each method determines a candidate breath rate and is performed concurrently on a computer system having a memory with instructions for executing the method. An improved breath rate is then determined in dependence on the determined candidate breath rate. Preferably, determining the improved breath rate includes using a statistical technique to compare a plurality of recognized breaths. Determining the improved breath rate can also preferably include determining reliability factors for individual breaths.

The present invention is also directed to a computer memory having instructions for executing the methods this invention; and also to a portable computing device including a handheld-type computing device operatively linked to a computer memory having instructions for executing the methods this invention. These instruction can further specify concurrently performing two or more instances of methods of this invention, the methods either being different or differently parameterized, and comparing breath occurrences recognized by the separate instances for reliability that recognized breath occurrences are true breaths so that reliable breath occurrences are output in dependence on the indicated reliability.

The present invention is also directed to a portable monitoring system for monitoring breath occurrences in a subject including size sensors, such as inductive plethysmographic sensors, disposed about the rib cage and/or abdomen of the monitored subject, wireless communications with a remote computer system, and a processing unit carried on or by the monitored subject operably linked to the size sensors, to the wireless communications, and to a memory. The memory of the portable system having instructions for performing one or more instances of any of the methods of this invention. When a plurality of methods are concurrently performed, these instruction further preferably compare breath occurrences recognized by the method instances to provide indicia of the reliability that recognized breath occurrences are true breaths so that breath occurrences can be output in dependence on the indicated reliability.

In methods of this invention recognizing R wave in ECG signals, the selected temporal relationship in which an R-wave can be recognized includes a time period having a start time and an end time, the start time being the occurrence time of the previous recognized R-wave plus a selected lockout period, and the end time the start time plus a selected searchable interval period. Here, the lockout period is between approximately 20% and approximately 50% of the median of the last seven R-wave intervals, and the searchable interval is between approximately ¾ and approximately 4/4 of the last R-wave interval in msec. Further in these methods, to determine the SNR, one moving average reflecting noise is sampled at approximately 400 samples or greater of the received ECG signal, and another moving average reflecting signal is sampled at approximately 24 samples or less of the received ECG signal. These parameters are for an ECG signal of approximately 200 Hz, the parameters for other sampling being proportionately adjusted.

In the methods and system of this invention, various of the method parameters, e.g., the selected duration and/or the selected fraction, are varied in dependence on subject activity, which preferably can be determined from one or more high-pass filtered accelerometer signals. Method parameters can also be downloaded to systems of this invention from remote computer systems. These remote systems can determine these parameters in real time in dependence on subject activity, or can select from pre-determined parameters also in dependence on subject activity.

This invention also includes embodiments having combinations of the methods and systems that, although not explicitly described herein, would be recognized by one of skill in the art to be useful and/or advantageous.

Thus, the present inventions describes systems and methods of extracting and determining real-time physiological signs from monitored data that overcome the disadvantages of the prior art.

### BRIEF DESCRIPTION OF THE DRAWINGS

The present invention may be understood more fully by reference to the following detailed description of preferred embodiments of the present invention, illustrative examples of specific embodiments of the invention, and the appended figures in which:

FIGS. 1A and 1B illustrate exemplary respiratory signals and their median filtering;

FIG. 2 illustrates the median, RSA, and combined methods;

FIGS. 3 and 4 illustrate results of exemplary methods for selecting median method parameters;

FIGS. 5 and 6 illustrate exemplary operation of threshold breath detection without activity level compensation and with activity level compensation, respectively;

FIG. 7 illustrates exemplary linear filter weights;

FIGS. 8A-F illustrate results of breath rate algorithms during various activities;

FIG. 9 illustrates the RSA phenomena and the operation of an exemplary RSA method;

FIG. 10 illustrates an embodiment of an R-wave determination algorithm;

FIG. 11 illustrates breath detection test data; and

FIG. 12 illustrates subject breath counting.

### DETAILED DESCRIPTION OF THE PREFERRED EMBODIMENTS

Preferred breath rate detection methods and systems are described herein, together with test data confirming their functioning. Headings are used for clarity of presentation and description of, but without limitation to, the invention as presented and described.

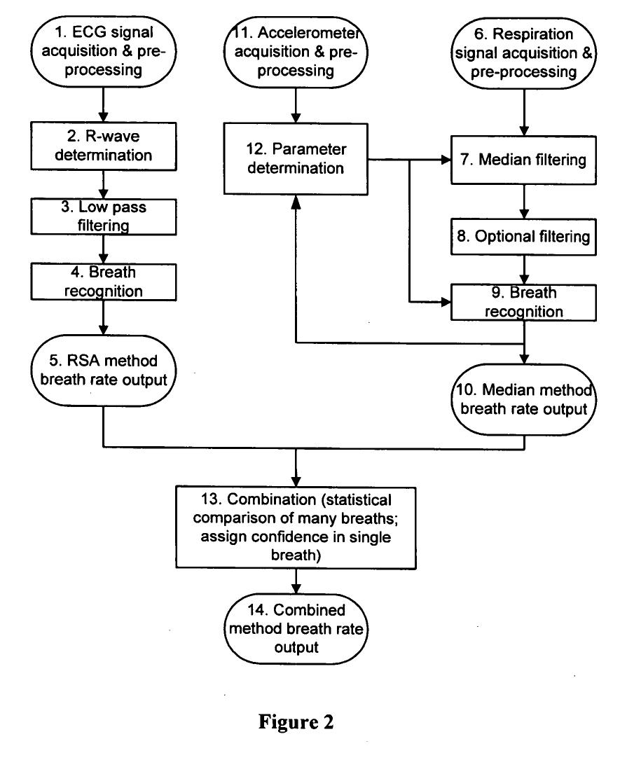

Referring initially to boxes 1 and 6 of FIG. 2, the present invention is applicable to respiratory signals arising from many known respiratory monitoring technologies. Solely for concreteness and compactness, and without prejudice, the invention is described herein largely in terms of respiratory signals arising from size sensors, and particularly from inductive plethysmographic (“IP”) “size sensors” preferably disposed about the rib cage and abdomen of a subject. Generally, “size sensors” gather signals responsive to various indicia of sizes of portions of a subject's body, such as the torso, the neck, the extremities, or parts thereof. Size sensors at one or more portions of the torso, e.g., at an abdominal portion and at a rib cage portion, provide indicia that can be interpreted using a two-component breathing model in order to determine respiratory rates, respiratory volumes, respiratory events, and the like.

This technology and associated methods of signal processing are described in the following U.S. patents and applications, which are incorporated herein in their entireties for all purposes and to which reference will be freely made: U.S. Pat. No. 6,047,203, issued Apr. 4, 2000, by Sackner et al.; U.S. Pat. No. 6,551,252, issued Apr. 22, 2003, by Sackner et al.; and U.S. patent application Ser. No. 10/822,260, filed Apr. 9, 2004, by Behar et al.

Additionally, motion and posture signals can be measured by accelerometers, and cardiac electrical activity signals can be measured by ECG electrodes.

### The Median Method

The invention includes two complementary and cooperative methods for breath rate determination, a median method and a respiratory sinus arrhythmia method.

Median Filtering

FIG. 2, boxes 6-10, generally illustrate the median method, which includes median filtering of signals derived from respiratory measurements and processing, followed by breath detection. Referring to box 12 of FIG. 2, parameters defining these steps must be carefully selected to produce suitable results in various applications of this invention. Also, the method includes various options and enhancements. The median method is now described with reference to processing of sample respiratory signals.

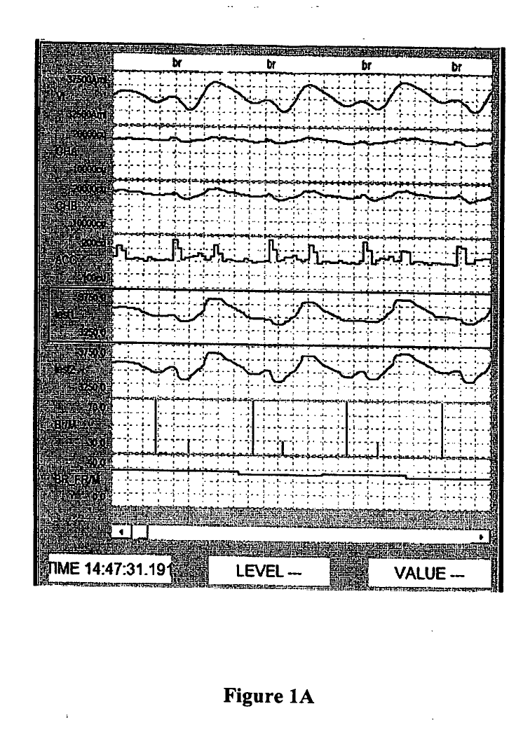

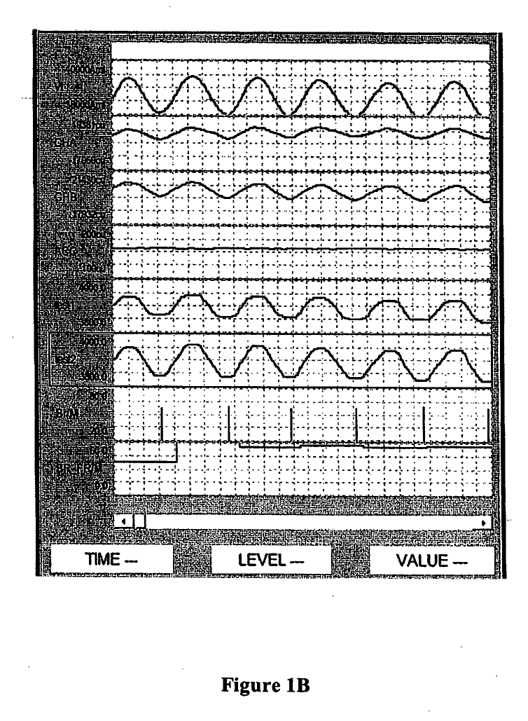

FIGS. 1A and 1B illustrate about nine seconds and six seconds, respectively, of respiratory signals typical of a particular application of this invention. These figures represent signals recorded during periods of more and less subject motion, respectively, and also present examples of their median filtering, and in both figures, the CHA and CHB traces represent measured changes in rib cage (“RC”) and abdominal (“AB”) sizes, which are combined according to a two-compartment breathing model to produce a trace representing tidal volume signal, the Vt trace. The ACC trace represents processed accelerometer signals. The test1 and test2 traces are results of median filtering that is further described below.

Turning to FIG. 1A, the Vt trace includes four relatively smaller and shorter local maxima and relatively four larger and longer local maxima. The larger and longer local maxima are respirations associated with actual movements of the RC and AB. Each respiration begins at a beginning of inspiration, which is the local minima lung volume just prior to a local maxima lung volume, and ends at the next beginning of inspiration, which is the next local minima prior to the next local maxima.

During these measurements, the subject was walking, and the ACC trace illustrates a number of short and sharp local maxima, which represent accelerations generated during walking (i.e., when the subject's foot contacts and/or leaves the ground). It can be clearly seen that the smaller and shorter local Vt maxima closely correlate with the short local maxima in the ACC trace, thereby identifying these local maxima as likely to be artifacts caused by subject motion and not by subject breathing.

Referring to box 7 of FIG. 2, these signals (breath signals including Vt and/or RC and/or AB signals) are first median filtered using a filter chosen and parameterized to largely remove such artifacts expected in a particular application of this invention. In this way, such motion artifacts are preferably not falsely identified as breaths.

Briefly, the median filtered value of a signal at a current time sample is preferably determined as the statistical median of a set of signal values at time samples surrounding and including the current time sample. Briefly, a median filter replaces a sample value with the median of the values of N nearby samples, usually the N/2 or N/2−1 time samples immediately subsequent to, i.e. in the future of, the current time sample, and the N/2 or N/2−1 time samples immediately previous to, i.e. in the past of, the current time sample. A median filter typically produces an output signal after a latency of about N/2 samples (N samples for the first signal value) in which short local maxima in the input signal are replaced with flatter regions or plateaus having a width of about one-half of the filter width in the output signal. Thus, a longer filter better removes artifacts in an input signal. However, a longer median filter can obscure physiologically significant components in an input signal. Alternately, a median filter can include N−1 past samples along with the current sample; such a median filter has no real-time latency.

Preferably, a median filter used for a particular embodiment of this invention is selected to be just long enough to filter the signal artifacts expected in the embodiment. In typical embodiments, undesired motion artifacts have a duration of about 200 msec to about 300 msec, as shown in FIG. 1A, the shortest filter that can be expected to provide for effective removal preferably has a length of about 400 msec to about 800 msec. The median filter used to generated the test2 trace in FIG. 1A has a length of 24 samples for a temporal length of about 480 msec, while the test1 trace has a length of 40 samples for a temporal length of about 800 msec. (The signals in FIGS. 1A and 1B were sampled at 50 Hz, or 20 msec per sample).

By comparing the test1 and test2 traces with the Vt trace, it can be seen that the test2 trace retains significant motion artifact, but of reduced amplitude and extended duration, while artifacts have largely been removed from test1 trace by the longer median filter used. Thus, a preferred median filter length, i.e., just lone enough to suppress artifacts expected in the monitoring environment of a particular embodiment, for this monitoring embodiment is no longer than about 50 samples or no longer than about 40 samples. In preferred embodiments, the length of the median filter is between about 30 samples and 40 samples so that artifacts are effectively removed with a shorter latency and less signal smoothing.

FIG. 1B illustrates how inappropriate median filtering may complicate breath detection, namely by causing reduction in amplitudes in the filtered Vt signal. As shown in FIG. 1B, the raw Vt trace has breath amplitudes of about 1450 ml, the test2 trace (where the median filter has length of 24 samples) has amplitudes of about 1230 ml, and the test1 trace (where the median filter has length of 40 samples) has amplitudes of about 850 ml. Reduction in amplitude with increasing median filter length is apparent. This reduction complicates breath detection because amplitudes of actual breaths become more similar to the amplitudes of signal background. Limiting amplitude reduction is a further consideration in the selection of appropriate median filter lengths.

Breath Detection

The median filtered signal is next examined for occurrences of recognizable breaths, as shown in box 9 of FIG. 2. In one embodiment, as shown in box 8 of FIG. 2, an optional additional linear filtering step may be applied to the median filtered signal prior to breath recognition in order to reduce higher frequency noise spikes by, e.g., noise with frequencies above the expected frequencies of breath signals (usually about 0.5-0.8 Hz or less).

The preferred breath detection method first scans a processed Vt signal and identifies occurrences of signal minima and signal maxima identifiable above any noise present in the signal. Identified maxima and minima are recognized as breaths if their amplitude and period are greater than selected bounds. A signal maxima and minima having small amplitude and short period is likely to be noise, subject motion, or other artifact, and not a true breath. Breath identification bounds can be selected in various ways, for example, by a state machine. In one embodiment, signal maxima and minima are identified as true breaths when signal changes within a selected period, e.g., 60 msec, and/or exceed a selected amount, e.g., 0.5%. Another embodiment preferably selects breath identification bounds by determining a running indicator of recent signal noise power, e.g., as a standard deviation of the past N samples after a linear de-trending, and identifying an actual breath if a relative signal change exceeds a certain number of standard deviations (e.g. one, or two, or three).

A further embodiment selects breath identification bounds by applying a statistical measure (e.g. median, mode, average, or the like) to a determined number of immediately prior actual breaths. The bounds are then determined by the statistical measure of signal amplitude and temporal period. In a preferred embodiment, a median amplitude and duration is determined for at least about 5 prior breaths and at most about 30 prior breaths. More preferably, the median is for least about 10 prior breaths and at most about 20 prior breaths. A preferred embodiment includes a median of about twelve prior breaths, which has been found to be a useful threshold. Additionally, the threshold may be fixed or otherwise selected.

The threshold percentage is referred to herein as “MRVt”. If the threshold is too low, the number of artifacts that are mis-recognized as true breaths increases, while a large threshold increases the number of true breaths that are not recognized. A useful range for a MRVt has been found to be between a relative value of about 5% and about 25%, after taking into account amplitude reduction by median filtering. An MRVt of about 5% is a practical minimum. For example, if the average recent breath is 2 liters, a 5% threshold identifies deviations above 100 ml as a breath. However, it is known that volumes of less than 100-200 ml ventilate only airways and not lungs. Preferably, MRVt can be adjusted automatically in a manner to be described.

It is generally less preferred to require minimum breath durations (or other fixed-breath timing characteristics) below which a signal deviation will not be recognized as a breath because breath timing and duration are known to vary significantly. Returning to FIG. 1B, the CHA and CHB signals indicate relatively steady breathing, and the ACC signal indicates little subject motion. However, even in these monitoring conditions, it can be seen that some breaths have durations down to less than about 1 sec. Similarly, during intense exercise at high breath rates, duration and frequency of true breaths can vary substantially from short to long.

Parameter Estimation

Preferred embodiments for estimating method parameters systematically and automatically select those parameters resulting in suitable breath detection performance, often expressed as a criteria that trades-off the number of actual breaths that are not detected versus the number of false breaths (i.e. artifact signals) that are detected as breaths. Because detection performance criteria and desired level of detection performance may vary for different applications or embodiments, preferred method parameters will advantageously vary accordingly. Described herein is an embodiment of a systematic estimation method that selects median filter length and MRVt to meet a common performance criteria, namely a maximum number of detected true breaths and a minimum number of detected false breaths. This method is illustrated using preferred indices of missed breaths and false breaths to estimate method parameters. Other embodiments can use other error indices that similarly provide information on missed or mis-detected breaths

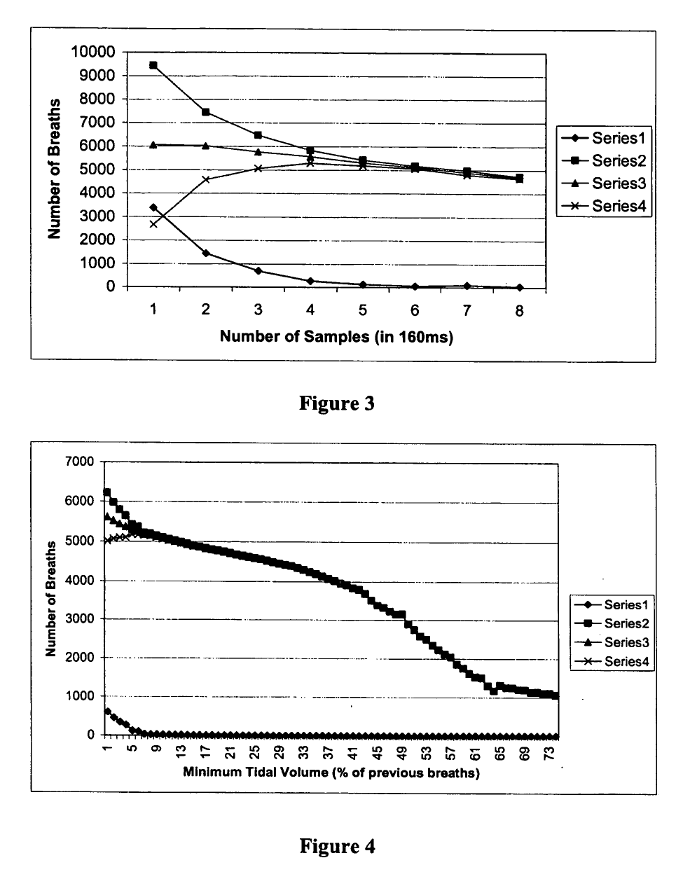

This parameter estimation method, shown in box 12 of FIG. 2, is illustrated with reference to the data presented in FIGS. 3 and 4. FIG. 3 illustrates four different indices of breath detection performance as curves labeled Series 1, 2, 3, and 4. The x-axis (labeled “Number of samples (in 160 msec)”) is the temporal median filter duration expressed as multiples of 160 msec. For example, an x-axis value of “5” indicates a 800 msec filter length. FIG. 4 illustrates breath detection indices, where the x-axis (labeled “Minimum Tidal Volume FIG. 4 (% of previous breaths)”) is MRVt as a percentage of the median of the previous twelve detected breaths. Series 1, 2, 3, and 4 have the following meanings: Series 1 is an estimate of the number of false breaths detected, where false breaths are considered to be those with a duration <1 s (even though such false breaths may in fact be actual breaths as described above); Series 2 is the total number of breaths detected in the Vt signal by the median method; Series 3 is an estimation of the number of non-artifact breaths as the difference of (Series2)−(Series 1); and Series 4 is an estimation of the number of true breaths determined as the difference of (Series2)−((Series 1)+(Series 1)), where the number of real breaths not detected is considered to be equal to the number of false breaths detected, and therefore the total number of detection errors is (Series 1)+(Series 1).

The systematic method illustrated by the exemplary data of FIGS. 3 and 4 selects parameters so that a maximum number of true breaths is detected, where this maximum is estimated as the number of detected breaths (Series 1) minus the number of breath detection errors (Series2 or 2*(Series 2)). Accordingly, parameters are preferably selected to maximize Series 3 and/or 4. Concerning median filter length, as shown in FIG. 3, Series 3 and Series 4 have a broad maximum for median filter lengths between about 640 msec and about 800 msec. Concerning MRVt, as shown in FIG. 4, and in particular comparing artifact breaths of Series 1 with Series 3 and/or Series 4, it is seen that MRVt should preferably be as small as possible to increase breath detection. If MRVt is less than about 5%, however, then most of the increase in detected breaths is due to mis-detected artifacts. Since Series 3 and Series 4 only slowly decline from about 5% to about 25%, a larger MRVt value is also reasonable to insure minimum mis-detection errors. For example, an MRVt of about 10% lowers the number of real breaths by only about 5% from about 5585 to about 5321. MRVt thresholds should be adjusted as median filter length changes because shorter or longer median filters can increase or decrease breath amplitudes in the filtered Vt signal.

Accordingly, in this particular embodiment, automatic parameter selection selects a preferred median filter length of about 40 samples and a preferred MRVt of about 5%. Although these parameter values are suitable for the particular embodiment illustrated and the particular selection criteria chosen, they may not be suitable for other test data and other criteria. However, the same automatic technique may be applied in other embodiments to determine other suitable sets of parameters. Additionally, different sets of parameters may be appropriate even for a single embodiment when a monitored subject engages in different activities or postures. Thus, additional parameters can be selected from predetermined sets of parameters in view of activity and posture data processed from accelerometer signals. It should be understood that the present invention includes these alternatives.

This invention also includes downloading method parameters from a server system with which a local handheld-type computer running the methods of this invention is in communication. In various embodiments, method parameters can be pre-computed according to the described methods and stored for later downloading. Alternatively, parameters can be determined in near real-time from monitoring data reported by the handheld-type computer. Parameters, whether pre-computed or determined online, can be automatically selected and/or selected or adjusted by monitoring personnel at the server system.

Breath Detection Enhancements

The previously described filtering and detection methods with fixed parameters are suitable in more predictable environments, where, for example, the intensity of subject motion is known or measured in advance and may be used for the generation of parameter estimation data. However, fixed parameters may not be suitable in other less predictable environments, where the intensity of subject motion can change from moment-to-moment. In these latter environments, distinguishing real breaths from motion artifacts becomes more difficult. For example, a median filtered signal will begin to pass artifact if the artifact becomes so prevalent that it contaminates a major fraction (e.g. about 50%) of the data samples within the filter length. Additionally, if the MRVt is set to about 5% in order to minimize missed breaths, these unfiltered artifacts will be detected as breaths and the breath rate signal will become unreliable.

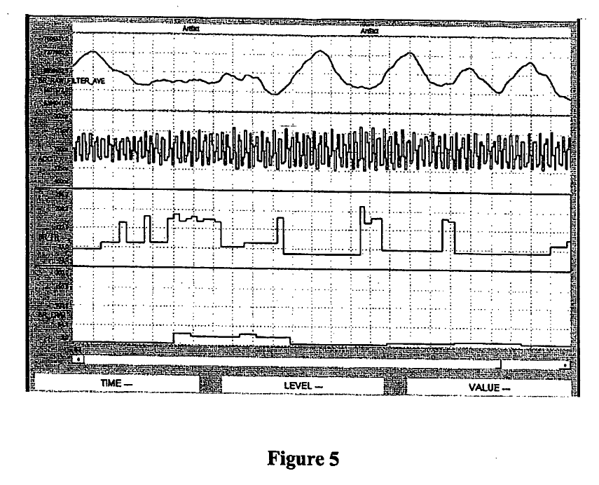

FIG. 5 illustrates this difficulty during 30 sec of respiratory and accelerometer data from a subject with a relatively small total lung volume who is running in place. A smaller lung volume makes true breaths even less apparent in comparison to the motion artifacts. The raw Vt signal, which is presented in the first trace, shows considerable irregularity, often obscuring respiratory activity due to the intense subject activity revealed in the ACC signal, which is presented in the second trace. Output of the median method using the above fixed parameters is illustrated in the third trace. The determined breath rate signal indicates an unusually high baseline breath rate of about 35 breaths/sec on which is superimposed spikes to entirely unreasonable breath rates of up to about 150 breaths/sec. Accordingly, the breath detection output must be considered unreliable at best, and likely simply wrong.

It has been discovered that performance of the median method can be enhanced in this and comparable situations by selected enhancements: first, the MRVt parameter is varied with subject activity level, which is measured by a motion index (“MI”); and second, the signal is additionally linearly filtered. The first enhancement generally increases MRVt as subject activity increases as indicated by the MI indicia. An MRVt of about 5% has been found suitable for periods of low activity, as described above. For periods of high activity, MRVt preferably increases, but in view of FIG. 4, preferably remains bounded at any activity level in order to avoid missing an excessive number of true breaths. If MRVt were not bounded, up to about 80% of true breaths may be missed, which is an unacceptable error rate. A suitable upper bound has been found to be about 25%, which remains on the slowly decreasing portions of the Series 2 and Series 3 curves in FIG. 4. Bounds other than about 5% and about 25% may be more suitable for other monitoring environments and/or other monitored subjects.

In more detail, a MI is determined from accelerometer signals and MRVt is preferably adjusted by scaling MRVt between its bounds in dependence on non-linear scaling of accelerometer signal intensity into a bounded range (i.e. the MI). First, MRVt is linearly adjusted between its bounds, for example about 5% to about 25%, according to the determined MI. The following equation has been found suitable:
MRVt=(5%)+(20%)*(MI/128).

Second, MI is determined by scaling accelerometer signal power determined from a monitored subject, which has a large range of values, into a bounded range, e.g., from about 0 to about 127. The scaling is preferably linear over the broadest possible power sub-range, but preferably becomes non-linear at high accelerometer signal levels so that all powers values are represented somewhere within the scaling range. A substantially logarithmic high-signal scaling has been found suitable. Since the signals scaled should primarily reflect the intensity of subject motion, input accelerometer signals are high pass-filtered to remove lower frequency, primarily postural components, while retaining higher frequency, primarily motion components, and are also converted from amplitude to power or intensity. The following code illustrates an embodiment of MI determination from input accelerometer signals, where ACCx and ACCy are raw signals from a two axis accelerometers sampled at 10 Hz:

```
long Acc[ ] = {w1, w2, w1};
dACCx = ACCx(filtered) − ACCx(unfiltered)
dACCy = ACCy(filtered) − ACCy(unfiltered)
MI_raw = (1/10)*sum (dACCx*dACCx)+sum(dACCy*dACCy)
MI = rescaleMotion(MI_raw)
int rescaleMotion(Int16 nMotion)
{
if (nMotion<100)
    { return nMotion; }
  else if (nMotion<1000)
    { return 100+(nMotion−100)/90; }
  else if (nMotion<10000)
    { return 110+(nMotion−1000)/900; }
  else return 127;
}
```

Here, dACCx and dACCy primarily contain higher frequency subject motion components since they are derived as the difference between unfiltered accelerometer signals and accelerometer signals filtered by a three-point low pass filter. dACCx and dACCy are then converted from amplitude to intensity (i.e. power) for resealing by procedure rescaleMotion. Since the accelerometer power has most often been found to be in the range of about 0 to about 100, this range is linearly scaled to an equal range of MI values from 0 to 100. Power values from about 100 up to the largest values are then logarithmically scaled into the remaining range of MI values, i.e. from 100 to 127. MI is then used to linearly adjust MRVt, as previously described. Other environments and subjects may benefit from more sophisticated accelerometer signal scaling procedures and MRVt adjustment, and in particular, the procedures may be combined into a single procedure that directly adjusts MRVt in dependence on input accelerometer signals.

In further embodiments, scaling of accelerometer power signals is differently selected or adjusted to reflect different monitoring environments. For example, in environments where activity is expected to be more intense, can compress low accelerometer power values into the lower portion of the scale range so that expected power signals occupy more of the scale range and method parameters can be more accurately selected,

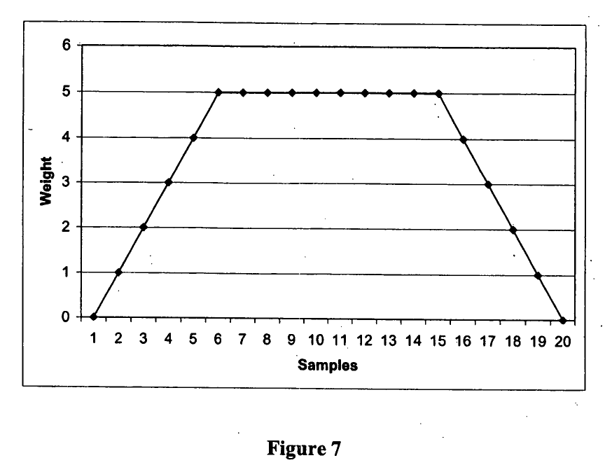

Another breath detection enhancement includes a linear FIR filter placed after median filtering and before breath detection. This filtering step can further attenuate signal artifacts, however, care should be taken to minimize smoothing or further amplitude reduction of the Vt signal. A preferred linear FIR filter preferably includes suitable filtering performance (for example, one that does not pass higher frequency artifacts) with a length equal to about half of the median filter length and with filter weights chosen for computational efficiency. Using a FIR filter length of about half of the median filter length advantageously smoothes the curve without further reducing the tidal volume signal. FIG. 7 illustrates exemplary relative weights for a length 20 FIR filter chosen so that an input signal can be filtered with only addition and subtraction operations, multiplication operations not being needed.

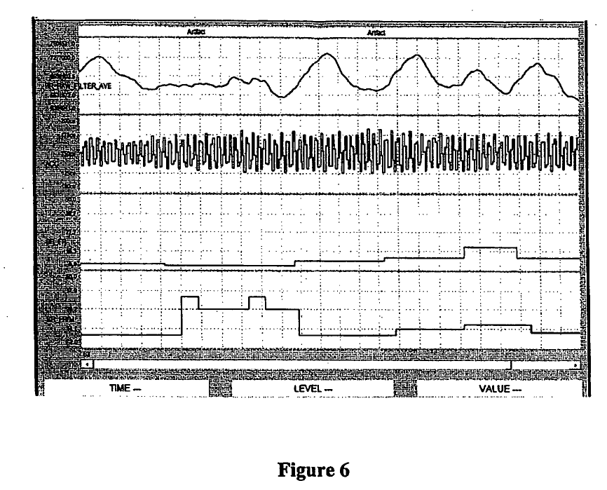

A comparison of FIG. 5 and FIG. 6 demonstrates the improvement due to these enhancements. The figures illustrate the same 30 sec respiratory and accelerometer data processed by the median method without the above-described enhancements (FIG. 5), and by the median method with the above-described enhancements (FIG. 6). The detected breath rate by the enhanced median method has a more normal baseline (i.e. about 10 breaths/min) and is free of superimposed spikes or entirely unreasonable breath rates. Instead, the detected breath rate gradually increases during exercise from a baseline rate of about 10 breaths/min to more typical increased rate of about 20 breaths/min.

Examples and Error Estimation

In addition to detecting and calculating breath occurrences and breath, the present invention also preferably includes estimating the reliability, or error, of the calculated breath and breath rate values. Preferably, these values are estimated by determining the sensitivity of breath rate based on variations of MRVt and the median filter width N.

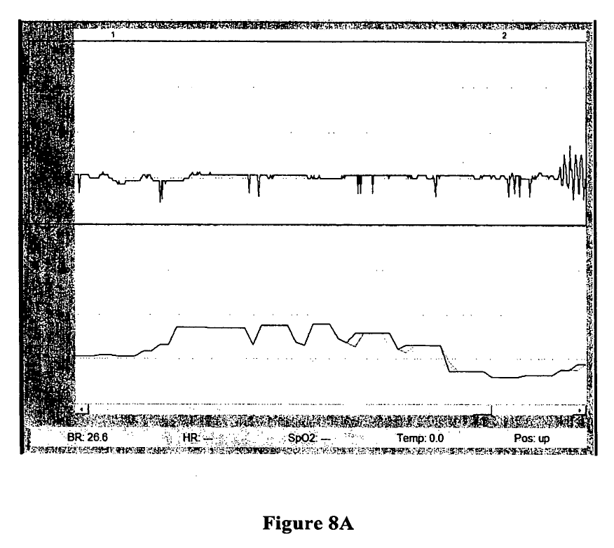

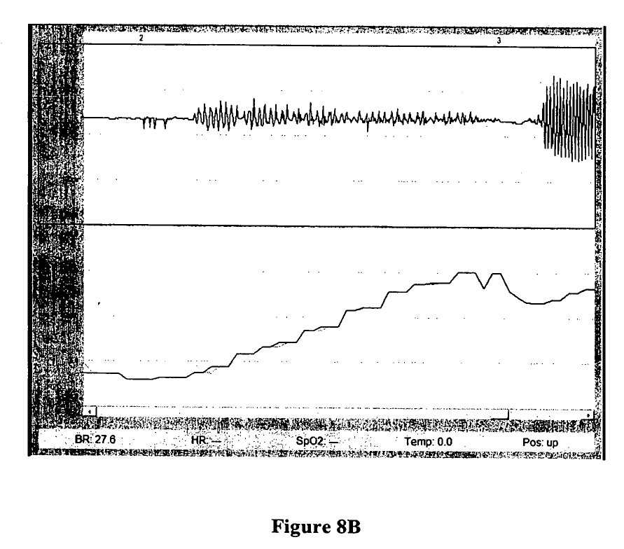

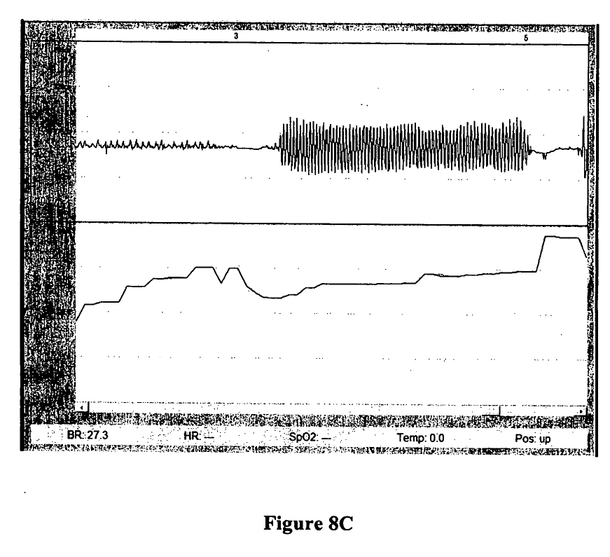

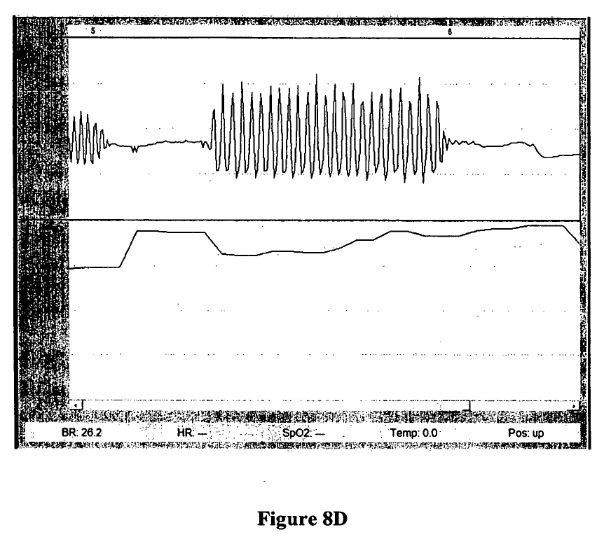

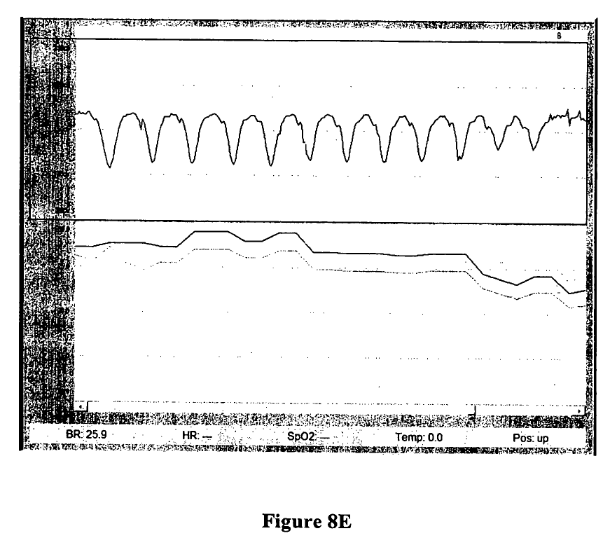

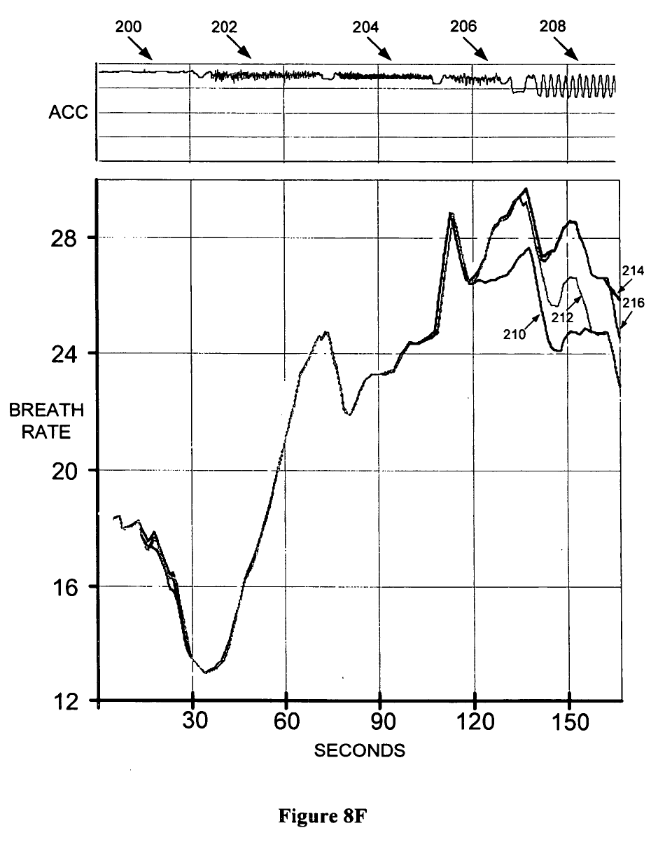

In one embodiment, the error is estimated by running the same breath rate algorithm six times with six different sets of parameters. For example, the six sets of parameters may include: 1) a master set where MRVt =15% and N=40 (not shown in FIGS. 8A-E); 2) a second set where MRVt=5% and N=40 (shown in green in FIGS. 8A-E); a third set where MRVt=25% and N=40 (shown in green in FIGS. 8A-E); a fourth set where MRVt=15% and N=32 (shown in yellow in FIGS. 8A-E); fifth set where MRVt=15% and N=48 (shown in yellow in FIGS. 8A-E); and a sixth set where MRVt=is motion dependent and N=40 (shown in black in FIGS. 8A-E). Sets 2 and 3 are preferably used to gauge the range of breath rate as a function of MRVt varying between 5% and 25%. Sets 4 and 5 are preferably used to gauge the range of breath rate as a function of median filter width N varying between 32 and 48.

Advantageously, the resulting five outputs of sets 1 to 5 can be used to assess the reliability of the tested algorithm, and the output of set 6 provides the best estimation of breath rate. These six sets of parameters, and the resulting breath rates, were tested during five different activities: 1) standing still (as shown in FIG. 8A); 2) walking (as shown in FIG. 8B); 3) running in place (as shown in FIG. 8C); 1) jumping jacks (as shown in FIG. 8D); and 5) forward folds (as shown in FIG. 8E).

As is seen in the ACC traces for each of FIGS. 8A-D, these activities produce a relatively higher frequency signal, and because the expected noise in Vt for these activities is well below 80 Hz, such noise is effectively removed by the median filter. Thus, the breath rate results of parameter sets 2-6 largely coincide, showing relatively good reliability of the breath rate algorithm.

FIG. 8E shows changes in the shape of the chest and/or abdomen during bending in the forward direction or in any other direction. Any activity of this kind happens on a time scale larger than 1 second and is often correlated with breathing such that it is difficult to remove via any filtering without running the risk of removing a true breath. In such a situation, the result of the algorithm becomes more sensitive to the threshold and filter parameter. The forward bending activity produces ACC traces with a relatively lower frequency signal, and thus any associated noise is expected to have a rate that is lower than the median filter frequency. While the resulting trace may be contaminated with motion artifacts due to reduced filtering efficiency, and thus causing the results of parameter sets 2-6 to diverge slightly, the breath rate reliability is still relatively good.

FIG. 8F presents similar parameter comparison data in an overlapped format. This figure includes a trace of breath rate versus time accompanied with a corresponding trace of accelerometer signal power; the breath rate trace has an enlarged vertical scale and the accelerometer trace has a reduced vertical scale. During period 200, the monitored subject was standing still; during period 202, the subject was walking; during period 204, the subject was running in place; during period 206, the subject was performing jumping jacks; and during period 208, the subject was performing forward folds. These data were analyzed using five sets of parameters. In trace 212, the median filter length was set at its upper threshold (see above); and in trace 210, MRVt was set at its upper threshold. In trace 214, the median filter length was set at its lower threshold (see above); this trace is coincident with and overlays a trace where MRVt was set at its lower threshold. Finally, in trace 216, parameters were adjusted from moment-to-moment by the previously-described adaptive process. All traces are 30 second moving averages.

Examining this figure, it is seen that even during the jumping jack and the forward fold activities, the different parameter sets produced breath rates that agreed within about ±5-6%. While over most of the activity range, the different parameter sets produced indistinguishable breath rate results. Comparing the different traces, it can also be seen that among the parameter sets, the most consistent results were produced by adaptively-setting the parameters or by setting median filter length and/or MRVt at or near lower thresholds. Setting these parameter at or near their upper limits, as in traces 210 and 212, underreported the breath rate at higher activity levels. This is expected because these traces result from greater median filtering or greater detection thresholds.

FIG. 8F, and FIGS. 8A-E, demonstrate that the methods of this invention produce reliable and consistent breath rate outputs over most types of subject activity. Further, with adaptive parameter selection or with appropriate fixed parameter selection, these methods produce reliable and consistent breath rate outputs even for intense subject activity of this kind.

### The RSA Method

RSA refers respiratory sinus arrhythmia, which is the variation of heart rate that occurs during the course of a respiration (e.g., from one beginning inspiration to the next beginning inspiration), and is found in many subjects, usually in younger more healthy subjects. RSA may be a major, or even dominant, component of short term heart rate variability (“HRV”). In view of the RSA effect, breath occurrences and a breath rate can be determined by examining a heart rate signal for minima and/or maxima indicating individual breaths, as shown generally in boxes 1-5 of FIG. 2.

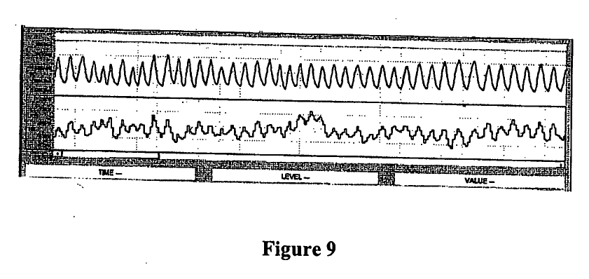

FIG. 9 presents 220 sec of signal illustrating RSA occurring while a subject is exercising. The first trace is the Vt signal, and the second trace is a concurrent heart rate signal. It is readily apparent that each breath in the Vt trace coincides with a periodic deviation in the heart rate trace such that peaks of lung volume (ending inspiration) closely correspond and are in phase with the peaks in heart rate. It is also apparent that the heart rate deviation due to coincident breaths account for most of the shorter period (i.e. higher frequency or “HF”) components of HRV. The remaining HRV components are readily distinguishable and of longer period (i.e. lower frequency or “LF”). It can be seen that, in these signals, breath occurrences can be easily and reliably determined from heart rate.

Referring back to box 1 of FIG. 2, the RSA method preferably proceeds as follows, beginning first with ECG signal acquisition and pre-processing to limit the effects of artifacts. Artifacts may arise in a heart rate signal from several causes. Strenuous motion can cause short duration artifacts that can be mis-identified as R-waves. Imprecise determination of R-wave occurrence times can lead to coordinated errors in adjacent heart rate values. Finally, ectopic heart beats, which occur intermittently in some subjects, can similarly cause errors in adjacent heart rate values. Therefore, artifacts can distort the heart rate signal and possibly introduce spurious maxima and minima. Accordingly, as shown in box 2 of FIG. 2, it is first preferred that a heart rate signal used in the RSA method be determined from two or more ECG electrodes to minimize motion artifacts. R-wave occurrences are preferably determined by the R-wave determination algorithm described below. In other embodiments, other reliable R-wave determination methods can be used, such as the known Pan-Tompkins algorithm. It is further preferred that ectopic R-waves be discarded, or optionally replaced by virtual R-waves which can be interpolated at the time the ectopic beat should have occurred in view of the local heart rate. Ectopic R-waves may be identified as R-waves occurring at a time that is more than a threshold duration before or after the R-wave occurrence time expected in view of the local heart rate (i.e., either too close to either a true prior R-wave or too close to the subsequent true R-wave).

Next, the heart rate signal is preferably filtered, as shown in box 3 of FIG. 2, to remove very short heart rate minima, and to enhance HF HRV relative to LF HRV. In particular, heart rate minima that occur within about 2 heart beats of each other are considered to be artifact, and are filtered out by using a simple linear filter such as:
HR(filtered)=¼*[HR(previous)+2*HR(current)+HR(next)].
Also, HF HRV may be enhanced by linear de-trending of the heart rate signal over short intervals, such as about three to about six breath times.

Finally, the pre-processed heart rate is examined for local minima and their immediately following local maxima by known signal processing means, as shown in box 4 of FIG. 2. Each local minima-local maxima pair then indicates a breath occurrence. Alternatively, local minima and/or local maxima alone may indicate breath occurrences. The breath rate may be determined for these indicated breath occurrences.

Method for R-Wave Determination

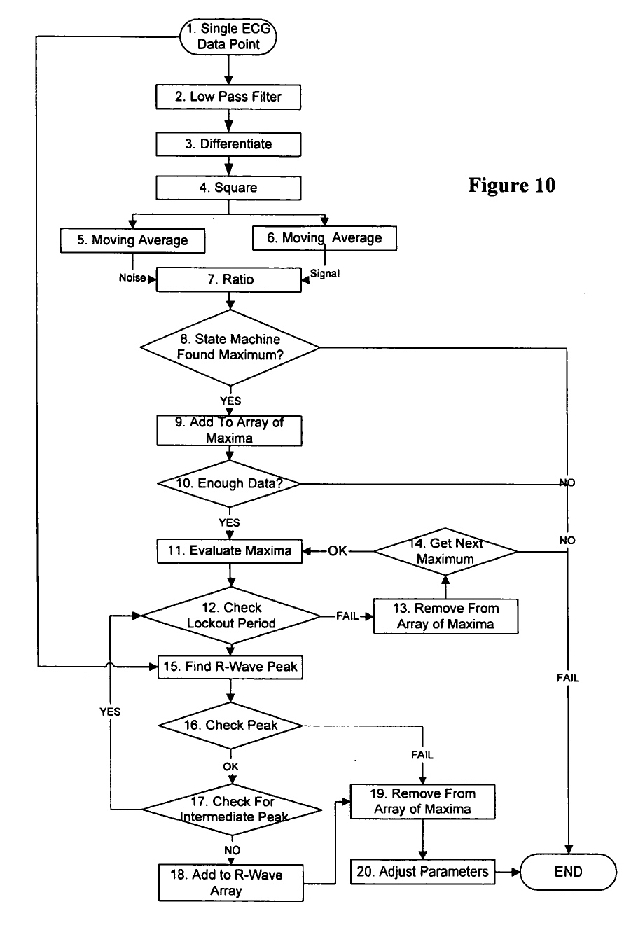

An embodiment of a preferred algorithm for identifying R-waves is shown in FIG. 10. The preferred algorithm has low latency so that it can be incorporated in a real-time system. Additionally, the algorithm preferably requires lesser CPU resources so that it can run on a hand-held PC in parallel with other algorithms. Advantageously, such a system runs multiple instances of this R-wave algorithm differing only in parameter selection and compares the outputs of the copies of the algorithm to select R-wave occurrences having increased confidence and reliability.

Referring to FIG. 10, the algorithm causally processes entirely a single ECG data point at a time (in view of prior processing of previous data points), as shown in step 1. Since the method is causal, latency is minimized. First, the signal is low-pass filtered, as shown in step 2, to smooth the curve for subsequent differentiation. Preferably, the filer order is 4. The signal is then differentiated in step 3 and squared in step 4.

Next, moving averages are established for both background noise and signal. In step 5, a four second moving average for background noise is computed, preferably with filter weights as shown in FIG. 7 and scaled to 800 samples (assuming a 200 Hz sampling rate) (alternatively 700, or 600, or 400, or 400 samples, or fewer). In step 6, a four second moving average for signal is computed, preferably with filter weights as shown in FIG. 7 and downsampled or scaled to 4 samples (alternatively 8, or 16, or 20, or 24 samples, or more). From these moving averages, a signal to noise ratio (“SNR”) is computed in step 7.

In step 8, the algorithm determines if a location (i.e. the beginning and end) of a potential R-wave has been identified. This is preferably performed by a state machine. Preferably, the beginning of a R-wave is found when the SNR exceeds a threshold SNR (“T(SNR)”), for example T(SNR)=2. Similarly, the end of a R-wave is found when the SNR drops below the T(SNR). A parameter is preferably used to describe whether the current value lies within a potential R-wave or not. A potential R-wave is found if the state of this parameter changes from true to false. This process is known as state machine. If a potential R-wave is found, it is added to an array of maxima (“AM”) log, which keeps track of beginning and end times when the SNR exceeds the T(SNR), in step 9.

Step 10 checks if enough date has been acquired. Preferably, the current time is checked to see if it is larger than the sum of the time of the last R-wave (“RW(last)”), the searchable time interval (“SI”), and the lockout period (“LP”) time interval. The SI is the time interval allotted for searching for the next R-wave candidate. One or two candidate R-waves can be located, but preferably not more than two in a single SI. Preferably, SI=k*RR(last), where the constant k= 5/4 (alternatively, ¾ or less, or 4/4, or 6/4, or 400 samples) and RR(last) is the last R-wave interval in msec. The LP is the minimum time interval between two consecutive R-waves. The LP preferably ranges between a lockout minimum and a lockout maximum, and is used to avoid R-wave misidentification. The LP preferably is a fixed percentage, for example 40% (alternatively 20% or less, or 30%, or 50%), of the median of the last seven R-wave intervals. The current time is also preferably checked to see if it is larger than the end time of the first maximum the AM or LP.

If enough data has been acquired, the next step in the algorithm is to evaluate the data, as shown in step 11. Initially, the algorithm searches for the next good R-wave in the SI. Preferably, the first maximum of the AM that exceeds the R-wave threshold (“T(R)”) is selected. T(R) is preferably 50% of the median threshold of the last seven R-wave intervals. If no such maximum is found in the AM, then the largest maximum in the SI is selected. If there is no such maximum in the SI, then the first maximum in the AM is selected.

In step 12, the LP is checked to see if there is a larger maximum in the LP. If there are two R-wave candidates in the LP, the candidate with the larger SNR is kept while the other is discarded. If the LP contains a larger maximum, then a next maximum is selected from the AM, as shown in step 14.

The beginning and end of the R-wave candidate is now identified. In step 15, the R-wave peak is preferably identified as the interpolated maximum of the raw, unprocessed ECG data. Raw data is stored in a short ECG cache until used in this step, and then discarded after use. The identified peak is checked in step 16 to confirm that it is an actual peak, rather than a discontinuity in the ECG signal. Step 17 checks for an intermediate peak, i.e. a maximum between the last R-wave peak and the current R-wave candidate. If no intermediate peaks are found, the R-wave candidate is added to the array of actual R-waves, as shown in step 18. Steps 19 and 20 include removing any R-wave candidates and all preceding maxima from the AM, and updating the filters and adjusting the parameters as described. The method then outputs R wave occurrences.

### Combined Methods

According to the present invention breath rate detection can also preferably include execution of two or more computationally-efficient breath rate detection methods followed by determination of a likely breath rate in dependence on the results returned from individual methods. The two or more methods can be different methods based on different principles, or differently-parameterized copies of one method, or a combination. This embodiment is advantageous because it permits in a more variable monitoring environment, where there is no single computationally-efficient breath rate detection method, which produces sufficiently reliable results over the range of expected monitoring conditions.

The concurrently executed detection methods can be based on different detection principles. A preferred embodiment, and as shown in box 13 of FIG. 2, includes the above-described median method together with RSA method. Alternatively, two or more of the concurrently executed detection methods can be based on similar detection principles but differently parameterized for different conditions. One preferred embodiment includes multiple instances of the median method with parameters selected for different levels of subject activity. For example, a low activity median method parameterization may have a shorter median filter and a fixed MRVt, and may omit additional linear filtering. Alternatively, a high activity median method parameterization may use longer median filters, additional linear filtering, and a variable MRVt. Further, the median filter can be supplemented or replaced by other types of artifact removal, such as a filter for short breaths, where a short threshold optionally varies with motion.

In some embodiments, the likely breath rate may be determined by using statistical techniques, such as the mode, median, weighted average, and the like applied to a number of recognized breaths (either preceding or surrounding the current breath in time). Prior to determination, outlier values are preferably discarded. Alternatively, a reliability index can be assigned to every recognized breath identified by the various detection methods, and used to determine a best breath rate from among the candidate breath rates. The reliability index can preferably be determined for each detection method from selected combinations of one or more of activity level, inhalation depth, shape of wave, and the like. A simple such reliability index is simply the fraction of concurrently executing methods that recognized a particular breath. It is preferable that the total computational requirement of the detection methods used not exceed available capacity of, for example, a handheld-type computer.

### Systems

The methods of the present invention are preferably coded in standard computer languages, including higher level languages such as C++, or the like, or for greater efficiency, in lower level languages such as C, assembly languages, or the like. The coded methods are then translated and/or compiled into executable computer instructions which are stored in computer memories (or loaded across network connections or through external ports) for use by handheld-type and other computers. Computer memories include CD-ROMs, flash cards, hard discs, ROM, flash RAM, and the like.

A handheld-type electronic device or computer as used herein refers to a module of a size and weight so that it can be unobtrusively and without discomfort by a monitored subject. However otherwise referred to herein, a handheld-type device or computer is not limited a microprocessor device, but can also include devices in which the methods of this invention have been encoded in, e.g., FPGAs, ASICSs, and the like. A handheld-type device suitable for performing the method of the present invention will typically include a low power microprocessor or other computing element with RAM memory and optionally one or more of the following components: ROM or flash RAM program memory, hard disk, user interface devices such as a touch screen, ports to external signal sources and/or data networks, and the like. Such a device will also include interfaces and/or ports for receiving sensor signals and pre-filtering and digitization if necessary.

This invention's methods typically require fewer processing resources, and therefore handheld-type computers with more limited processor capabilities are also suitable for performing the method of this invention. The methods of the present invention may also be run on standard PC type or server type computers which typically have greater processor capabilities.

### EXAMPLES

The present invention is illustrated by the following examples that are merely for the purpose of illustration and are not to be regarded as limiting the scope of the invention or the manner in which it can be practiced.

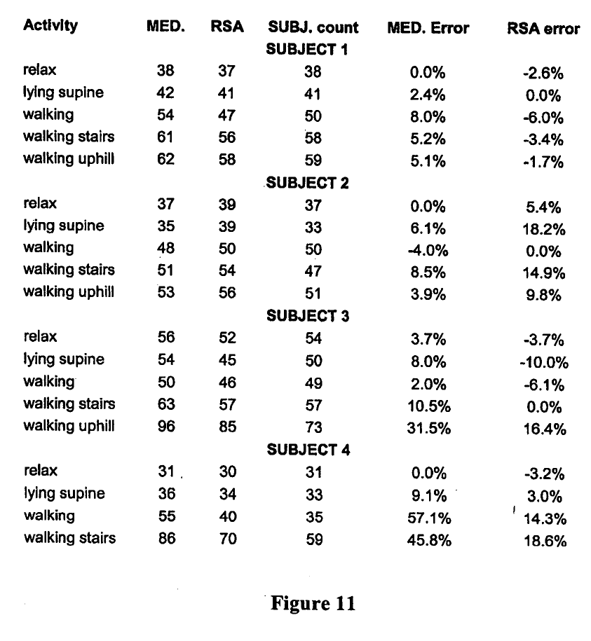

Respiration and accelerometer signals were gathered from four subjects performing selected activities ranging from no activity to walking uphill. Signals were processed according to the methods of the present invention and the results are presented in FIG. 11. The leftmost column (Activity) lists subject activities; the column second from left (MED.) lists the results of processing the gathered signals using the median method; the column third from left (RSA) lists the results of processing the gathered signals using the RSA method; the column fourth from left (SUBJ. count) lists the subjects' manual count of their breaths recorded by having the subjects press a handheld button; the column fifth from left (MED. Error) lists the percentage error between the breaths determined by the median method and the results of the subject breath count; and the column sixth from left (RSA error.) lists the percentage error between the breaths determined by the median method and the results of the subject breath count.

The last two columns of FIG. 9 (i.e. MED. Error and RSA error) show the relative error of the median method and the RSA method with respect to the subjects' own breath counts. It can be seen that in most cases, even for cases of more strenuous activity, both methods are accurate, with the median method being perhaps slightly more accurate than the RSA. However, for two subjects (i.e. SUBJECTS 3 and 4) and for more strenuous activity, both methods show considerable error.

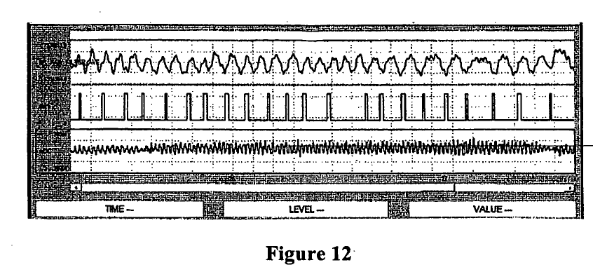

In this regard, it has been found that certain subjects may make considerable errors, generally by undercounting, when counting their own breaths. Counting breaths requires considerable concentration, which can be difficult to muster especially during periods of more strenuous exercise. FIG. 12 illustrates such undercounting, where the top trace shows about 70 sec of a raw tidal volume signal from an exercising subject, the bottom trace is the corresponding accelerometer signal, and the middle trace indicates when the subject indicated a breath by pushing a button. The subject counted 22 breaths during a period when there were 31 actual breaths, for a loss of about 30%. Many breaths were clearly not counted in the second half of this monitoring period. The monitoring data for SUBJECT 4 of FIG. 11 du ring walking similarly reveals that up to about 20 breaths were not counted, accounting for most of the 57% error. Accordingly, the available test data strongly suggest that both breath detection methods have an accuracy better than about 15%, and probably better than about 10%.

The term “about,” as used herein, should generally be understood to refer to both the corresponding number and a range of numbers. Moreover, all numerical ranges herein should be understood to include each whole integer within the range.

The present invention described and claimed herein is not to be limited in scope by the preferred embodiments herein disclosed, since these embodiments are intended as illustrations of several aspects of the invention. Any equivalent embodiments are intended to be within the scope of the present invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference for all purposes. Further, none of these references, regardless of how characterized above, is admitted as prior to the invention of the subject matter claimed herein.

What is claimed is:

1. A computer-implemented median method for recognizing occurrences of breaths in respiratory signals comprising:
receiving digitized respiratory signals having tidal volume information;
filtering the received respiratory signals to limit artifacts of a duration less than a selected duration; and
recognizing breaths in the filtered respiratory signals, wherein a breath is recognized when amplitude deviations in filtered tidal volume signals exceed a selected threshold fraction of an average of a plurality of previously recognized breaths.

2. The computer-implemented method of claim 1, further comprising determining a breath rate from the occurrences of recognized breaths.

3. The computer-implemented method of claim 1, wherein the selected threshold fraction varies in dependence on a subject activity level.

4. The computer-implemented method of claim 1, wherein filtering the respiratory signals comprises filtering at least one respiratory signal sample by taking a median value of a group of respiratory signal samples including the respiratory signal sample being filtered, all samples of the group occurring during the selected duration.

5. The computer-implemented method of claim 4, wherein filtering the respiratory signals further comprises linear low-pass filtering.

6. The computer-implemented method of claim 1, wherein the selected duration varies in dependence on a subject activity level determined from one or more high-pass filtered accelerometer signals.

7. The computer-implemented method of claim 1, further comprising an RSA method for recognizing breath occurrences including the steps:
recognizing R-waves in an electrocardiographic signal.
recognizing breaths from variations in the R-wave that are reflective of respiratory sinus arrhythmia.

8. The computer-implemented method of claim 7, wherein recognizing R waves further comprises:
determining a signal-to-noise (“SNR”) ratio by comparing two differently sampled moving averages of the received electrocardiogram (“ECG”) signal;
selecting signal maxima where the determined SNR exceeds a selected SNR threshold; and
recognizing an R-wave in the received ECG signal when a selected signal maximum occurs in a determined temporal relationship to adjacent recognized R-waves.

9. The computer-implemented method of claim 7, further comprising:
comparing breath occurrences recognized by the median method and breath occurrences recognized by the RSA method to provide indicia of the reliability that recognized breath occurrences are true breaths; and
outputting breath occurrences in dependence on the indicated reliability.

10. The computer-implemented method of claim 7, wherein the indicated reliability further comprises reliability indicia for each recognized breath occurrence.

11. The computer-implemented method of claim 1 further comprising:
concurrently performing at least one additional instance of the steps of receiving, filtering, and recognizing, wherein the selected fraction and/or the selected duration of the separate instances are different;
comparing breath occurrences recognized by the separate instances to ascertain the likelihood that recognized breath occurrences are true breath occurrences; and
outputting recognized breath occurrences in dependence on the indicated reliability.

12. The computer-implemented method of claim 1, wherein detecting the respiratory signals comprises using inductive plethysmographic size sensors disposed about the rib cage and/or abdomen of a monitored subject.

13. A computer memory having instructions for executing the median method of claim 1.

14. A computer system comprising a handheld-type computer operatively linked to a computer memory of claim 13.

15. The computer system of claim 14, wherein the computer memory further includes instructions for:
concurrently performing at least one additional instance of the steps of receiving, filtering, and recognizing, wherein the selected fraction and/or the selected duration of the separate instances are different;
comparing breath occurrences recognized by the separate instances for reliability that recognized breath occurrences are true breaths; and
outputting reliable breath occurrences in dependence on the indicated reliability.

16. A computer-implemented method for recognizing occurrences of breaths in respiratory signals comprising:
receiving a least one digitized size sensor signal reflecting respiratory motions of the rib cage and/or the abdomen of a monitored subject
determining a plurality of tidal volume (Vt) signal samples from the received respiratory signals;
filtering at least one Vt signal sample by taking a median value of a group of Vt signal samples including the Vt signal sample being filtered which occur during an interval having a duration less than a selected duration; and
recognizing breaths in the filtered respiratory signals, wherein a breath is recognized when amplitude deviations in filtered tidal volume signals exceed a selected threshold fraction of an average of a plurality of previously recognized breaths.
wherein the selected duration and/or the selected fraction vary in dependence on subject activity determined from one or more high-pass filtered accelerometer signals

17. The computer-implemented method of claim 16, further comprising:
recognizing R-waves in an electrocardiogram (“ECG”) signal.
recognizing breaths from variations in the R-wave rate that are reflective of respiratory sinus arrhythmia.

18. The computer-implemented method of claim 17, wherein R-wave signal recognition comprises:
determining a signal-to-noise (“SNR”) ratio by comparing two differently samples moving averages of the received ECG signal;
selecting signal maxima where the determined SNR exceeds a selected SNR threshold; and
recognizing an R-wave in the received ECG signal when a selected signal maximum occurs in a determined temporal relationship to adjacent recognized R-waves.

19. A computer-implemented method for recognizing R-waves in electrocardiographic signals comprising:
receiving a digitized electrocardiographic (“ECG”) signal;
determining a signal-to-noise ratio (“SNR”) by comparing two differently sampled moving averages of the received electrocardiogram (“ECG”) signal;
selecting signal maxima when ECG signal deviations exceed a selected SNR threshold; and
recognizing R-waves in the received ECG signal from when the selected signal maxima occur in a determined temporal relationship to adjacent recognized R-waves.

20. The computer-implemented method of claim 19, wherein the R-wave occurrences are filtered to remove minima therein.

21. The computer-implemented method of claim 19, further comprising recognizing occurrences of breaths in dependence on variation in the recognized R-waves.

22. The computer-implemented method of claim 19, further comprising:
receiving digitized respiratory signals having tidal volume information;
filtering the received respiratory signals to limit artifacts of a duration less than a selected duration; and
recognizing breaths in the filtered respiratory signals, wherein a breath is recognized when amplitude deviations in filtered tidal volume signals exceed a selected threshold fraction of an average a plurality of previously recognized breaths.

23. The computer-implemented method of claim 22, further comprising determining a breath rate from the occurrence of recognized breaths.

24. The computer-implemented method of claim 19, wherein the selected temporal relationship comprises a time period having a start time and an end time, the start time being the occurrence time of the previous recognized R-wave plus a selected lockout period, and the end time the start time plus a selected searchable interval period.

25. The computer-implemented method of claim 24 wherein the selected lockout period is the median of a plurality of previous R-wave intervals multiplied by a selected faction.

26. The computer-implemented method of claim 24 wherein the selected searchable interval period is a multiple of a mean of one or more previous R-wave intervals.

27. The computer-implemented method of claim 19 further comprising identifying R-wave peaks by interpolating the ECG signal as received.

28. A computer-implemented method for determining occurrences of breaths in physiological signals gathered from a monitored subject, the method comprising:
performing concurrently on a handheld-type computer one or more breath recognition methods, wherein each method recognizes candidate breaths; and
recognizing breath occurrences by comparing candidate breath occurrences.

29. The computer-implemented method of claim 28, wherein recognizing breath occurrences comprises using a statistical technique to compare a plurality of candidate breath occurrences.

30. The computer-implemented method of claim 28, wherein recognizing breath occurrences comprises determining reliability factors for individual candidate breaths.

31. The computer-implemented method of claim 30, further comprising a outputting reliability factor along with at least one recognized breath occurrence.

32. A portable system for monitoring breath occurrences in a subject comprising:
size sensors disposed about the rib cage and/or abdomen of the monitored subject;
wireless communications with a remote computer system;
a processing unit carried on or by the monitored subject operably linked to the size sensors, to the wireless communications, and to a memory having computer instructions for performing a median method of breath recognition including the steps of:
receiving at least one digitized size sensor signal reflecting respiratory motions of the rib cage and/or the abdomen of the monitored subject
determining a plurality tidal volume (Vt) signal samples from the received respiratory signals;
filtering at least one Vt signal sample by taking a median value of a group of Vt signal samples including the Vt signal sample being filtered, all signal samples of the group occurring during an interval having a duration less than a selected duration; and
recognizing breaths in the filtered respiratory signals, wherein a breath is recognized when amplitude deviations in filtered tidal volume signals exceed a selected threshold fraction of an average of a plurality previously recognized breaths.
wherein the selected duration and/or the selected fraction varies from time-to-time during subject monitoring.

33. The system of claim 32, wherein the memory further has instructions for:
receiving a digitized electrocardiogram (“ECG”) signal;
determining a signal-to-noise ratio (“SNR”) by comparing two differently sampled moving averages of the received ECG signal;
selecting signal maxima when ECG signal deviations exceed a selected SNR threshold; and
recognizing R-waves in the received ECG signal when the selected signal maxima occur in a determined temporal relationship to adjacent recognized R-waves.

34. The system of claim 33 wherein, the selected temporal relationship comprises a time period having a start time and an end time, the start time being the occurrence time of the previous recognized R-wave plus a selected lockout period, and the end time the start time plus a selected searchable interval period.

35. The system of claim 33, wherein the memory further has instructions for recognizing occurrence breaths in dependence on maxima of an R-wave occurrence rate.

36. The system of claim 34, wherein the memory further has instructions for:
comparing breath occurrences recognized from Vt signals and from ECG signals to provide indicia of the reliability that recognized breath occurrences are true breaths; and
outputting breath occurrences in dependence on the indicated reliability.

37. The computer-implemented method of claim 34, wherein the indicated reliability further comprises reliability indicia for each recognized breath occurrence.

38. The system of claim 32, wherein the memory further has instructions for:
performing concurrently at least one additional instance of a method recognizing breath occurrences;
comparing breath occurrences recognized by the plurality of methods for recognizing breath occurrences to ascertain the likelihood that recognized breath occurrences are true breath occurrences; and
outputting recognized breath occurrences in dependence on the indicated reliability.

39. The system of claim 32, wherein the memory further has instructions for varying the selected duration and/or the selected fraction in dependence on subject activity determined from one or more high-pass filtered accelerometer signals.

40. The system of claim 32, wherein the memory further has instructions for receiving values for the selected duration and/or the selected fraction from a remote computer system.

41. The system of claim 40, wherein the values received have been determined at the remote computer system in dependence on subject activity of the monitored subject determined from one or more high-pass filtered accelerometer signals.

42. The system of claim 34, wherein the lockout period is between approximately 20% and approximately 50% of the median of the last seven R-wave intervals

43. The system of claim 34, wherein the searchable interval is between approximately ¾ and approximately 4/4 of the last R-wave interval in msec.

44. The method of claim 33, wherein the sampling rate of the ECG signal is approximately 200 Hz, wherein a first moving average reflecting noise is sampled at approximately 400 samples or greater of the received ECG signal, and a second moving average reflecting signal is sampled at approximately 24 samples or less of the received ECG signal.
